(19) United States
(12) Patent Application Publication
(10) Pub. No.: US 2023/0147927 A1
(43) Pub. Date: May 11, 2023

(54) AIRWAY MANAGEMENT ISOLATION CHAMBER

(71) Applicant: The Government of the United States, as Represented by the Secretary of the Army, Fort Detrick, MD (US)

(72) Inventors: Nathan T. FISHER, Westminster, MD (US); Charles A. RILEY, Washington, DC (US); Joseph S. KRIVDA, Kensington, MD (US); Steven S. HONG, Alexandria, VA (US); Timothy C. BLOOD, Jr., Silver Spring, MD (US); Jonathan N. PERKINS, Rockville, MD (US); Douglas S. RUHL, DuPont, WA (US)

(73) Assignee: The Government of the United States, as Represented by the Secretary of the Army, Fort Detrick, MD (US)

(21) Appl. No.: 17/917,605

(22) PCT Filed: Apr. 9, 2021
(86) PCT No.: PCT/US21/26591

§ 371 Date: Oct. 7, 2022

Related U.S. Application Data
(60) Provisional application No. 63/008,033, filed on Apr. 10, 2020.
(60) Provisional application No. 63/009,885, filed on Apr. 14, 2020.

(51) Int. Cl. A61G 10/00; A61G 10/02; A61B 90/00

(52) U.S. Cl. CPC A61G 10/005; A61G 10/023; A61B 90/05

(57) ABSTRACT

An airway management isolation enclosure and a method for protecting medical personnel from infectious agents when treating a patient having an infectious disease, comprising: (a) a frame that is positioned over the head and anterior thoracic region of the patient; (b) a transparent barrier supported by frame, which covers and forms a seal around the head and anterior thoracic region of the patient and allows medical personnel to create sealable entry ports; and (c) an air flow entry and a vacuum exit connection to direct the infectious agents in the form of respiratory droplets and particles produced by the patient to flow away from the head of the patient and out of the transparent covering while the medical personnel manages the airway and upper aerodigestive tract of the patient.

This application claims priority to U.S. provisional patent application Ser. Nos. 63/008,033 filed Apr. 10, 2020 and 63/009,885 filed Apr. 14, 2020.

### SPONSORED RESEARCH AND DEVELOPMENT

The invention was made with government support from organizations within the Defense Health Agency and the United States Army Medical Research and Development Command. The United States Government has certain rights in the invention.

### FIELD OF INVENTION

This invention relates generally to an isolation chamber that allows medical personnel to safely and efficiently perform necessary airway and aerodigestive procedures on a patient suffering from an easily transmittable and infectious disease during treatment of the patient. More particularly, to an isolation chamber and methods for protecting medical personnel against exposure from infectious agents transmitted from patients, while performing such medical procedures.

### BACKGROUND OF INVENTION

COVID-19 is a virulent species of coronaviridae that often causes severe pulmonary damage to patients and is easily transmitted to healthcare workers. COVID-19 is typically caused by the spread of the virus in respiratory droplets and/or particles from an infected patient and the virus has been shown to persist in the environment for significant periods with up to 3 hours of viability in aerosols and up to 3 days on surfaces. Hospitalized patients with this infectious disease often require significant respiratory care and medical procedures associated with such care, such as nebulization and intubation. Case reports in China documented a severely increased risk for healthcare professionals managing the airways and upper aerodigestive tracts of these patients. In fact, current critical care algorithms advocate for early intubation which increases the demand for ventilators. Exposed healthcare providers often become infected more frequently as compared to their non-medical counterparts likely due to increased viral load from higher exposure.

It is desirable to isolate a patient having a highly contagious infectious disease to ensure that the infectious disease is not spread to the medical personnel providing treatment. Conventional isolation devices and structures exist in the art for isolating a patient to protect medical personnel against prolonged exposure to the infectious disease. Unfortunately, prior art isolation apparatuses currently available for treating the patient in the field provide only physical barriers. These physical barriers do not effectively prevent transmission of the infectious disease to the medical personnel performing medical procedures during treatment to the infected patient. Other isolation chambers, such as disclosed in U.S. Pat. No. 7,503,890, are isolation systems that are cumbersome and difficult for medical personnel to perform medical procedures such as nebulization or intubation to the patient during treatment.

One solution to this problem, as disclosed in the present invention, is to provide an isolation chamber that is placed over an infected/contagious patient's head to contain disease and prevent contamination of the environment and mitigate exposure to medical personnel and healthcare workers. In addition to a barrier, vacuum suction can be used to decrease the concentration of disease within the chamber, which is particularly helpful during nebulizer, bilevel positive airway pressure (BiPAP) and continuous positive airway pressure (CPAP) therapy and could prevent patients from needing a highly coveted respiratory ventilator.

### SUMMARY OF THE INVENTION

The present invention is directed to an airway management isolation enclosure that allows medical personnel to safely manage the airway and upper aerodigestive tract of a patient having a transmittable infectious disease, while isolating the infected patient to an area about a bed, wherein the infected patient is supine or sitting upright on the bed.

The invented airway management isolation enclosure for protecting medical personnel from infectious agents when performing airway management or other medical procedures on a patient having an infectious disease, comprises: (a) a frame that is positioned over the head and anterior thoracic region of the patient; (b) a transparent barrier supported by the frame, which covers and forms a seal around the head and anterior thoracic region of the patient and which allows the medical personnel to create sealable entry ports; and (c) an air flow entry and a vacuum exit connection to direct the infectious agents in the form of respiratory droplets and particles produced by the patient to flow away from the head of the patient and out of the transparent barrier while the medical personnel manages the airway and upper aerodigestive tract of the patient. The term “air flow” as used herein includes the use of other breathable gases, such as medical oxygen.

In one embodiment, the airway management isolation enclosure comprises a frame that is hollow and further comprises a plurality holes and further comprises one or more air flow entry connections and one or more vacuum exit connections to direct air flow away from the head of the patient and out of the transparent covering. In a separate embodiment, the airway management isolation enclosure comprises an additional frame that is hollow and further comprises a plurality holes, wherein the additional frame is proportionally and progressively smaller, each frame further comprising one or more air flow entry connections and one or more vacuum exit connections,

In a separate embodiment, the air flow entry and a vacuum exit connection are a separate unit that is supported on the frame of the airway management isolation enclosure. The air flow entry and vacuum exit are connected by flexible tubing. The frame supports tubing comprising a plurality of holes, an air flow entry connection and a vacuum exit connection, to direct air flow away from the head of the patient and out of the isolation enclosure.

In a separate embodiment, the transparent barrier comprises a disposable plastic material. In another embodiment, the transparent barrier comprises a reusable and sterilizable material, including metals and ceramics.

In a separate embodiment, the airway management isolation enclosure comprises a modular frame, wherein the frame comprises polyvinylchloride (PVC) and is sterilizable.

In a separate embodiment, the airway management isolation enclosure comprises a modular frame, wherein the frame comprises a stainless steel that is autoclavable.

In a separate embodiment, the airway management isolation enclosure protects medical personnel while performing airway management from infectious agents selected from viruses possess positive-sense single-stranded RNA genomes, including the picornaviruses (which is a family of viruses that includes well-known viruses like Hepatitis A virus, enteroviruses, rhinoviruses, poliovirus, and foot-and-mouth virus), SARS virus, hepatitis C virus, yellow fever virus, and rubella virus, viruses possess negative-sense single-stranded RNA genomes, including Ebola and Marburg viruses, influenza virus, measles, mumps, rabies, Virion-Type of naked/Capsid nucleic Virus Family Virus Genera enveloped Symmetry acid, Reoviridae Reovirus, Rotavirus Naked Icosahedral, Picornaviridae Enterovirus, Rhino virus, Naked Icosahedral Hepatovirus, Cardiovirus, Aphthovirus, Parechovirus, Erbovirus, Kobuvirus, Teschovirus Caliciviridae, Norwalk virus, Hepatitis E virus Naked Icosahedral, Togaviridae Rubella virus Enveloped Icosahedral, Arenaviridae Lymphocytic choriomeningitis Enveloped Complex virus, Retroviridae HIV-1, HIV-2, HTLV-I Enveloped Complex, Flaviviridae Dengue virus, Hepatitis C virus, Enveloped Complex Yellow fever virus, Orthomyxoviridae Influenzavirus A, Influenzavirus B, Enveloped Helical ss Influenzavirus C, Isavirus, Thogotovirus, Paramyxoviridae Measles virus, Mumps virus, Enveloped Helical Respiratory syncytial virus, Bunyaviridae California encephalitis virus, Enveloped Helical Hantavirus, Rhabdoviridae Rabies virus Enveloped Helical, Filoviridae Ebola virus, Marburg virus Enveloped Helical, Coronaviridae Corona virus Enveloped Complex, SARS-CoV-2, COVID-19, Astroviridae Astrovirus Naked Icosahedral, Bornaviridae Borna disease virus Enveloped Helical, and bacterial pathogens.

In one embodiment, there is provided a method for protecting medical personnel from infectious agents when performing airway management on a patient having an infectious disease by assembling an isolation enclosure that comprises the steps of: (a) positioning a frame over the head and anterior thoracic region of the patient laying in a supine position or a sitting position; (b) placing a transparent barrier supported by frame, which covers and forms a seal around the head and anterior thoracic region of the patient; (c) creating sealable entry ports in the transparent barrier; and (d) using a vacuum from an exit connection to direct the infectious agents in the form of respiratory droplets and particles produced by the patient to flow away from the head of the patient and out of the transparent barrier while the medical personnel manages the airway and upper aerodigestive tract of the patient.

### BRIEF DESCRIPTION OF THE DRAWINGS

The following drawings form part of the present specification and are included to further demonstrate certain embodiments of the present invention. The invention may be better understood by reference to one or more of these drawings in combination with the detailed description of specific embodiments presented herein.

FIG. 1 represents a conventional physical barrier to shield medical personnel from infectious agents during airway management of a patient.

FIG. 2 shows one embodiment of the invented airway management isolation enclosure for protecting medical personnel comprising: (a) a plastic frame that is positioned over the head and anterior thoracic region of the patient; (b) a transparent barrier supported by the frame, which covers and forms a seal around the head and anterior thoracic region of the patient and which allows the medical personnel to create sealable entry ports; and (c) an air flow entry and a vacuum exit connection to direct the infectious agents in the form of respiratory droplets and particles produced by the patient to flow away from the head of the patient and out of the transparent barrier while the medical personnel manages the airway and upper aerodigestive tract of the patient.

FIG. 3 shows a separate embodiment of the invented airway management isolation enclosure for protecting medical personnel comprising: (a) a metal frame that is positioned over the head and anterior thoracic region of the patient; (b) a transparent barrier supported by the frame, which covers and forms a seal around the head and anterior thoracic region of the patient and which allows the medical personnel to create sealable entry ports; and (c) an air flow entry and a vacuum exit connection to direct the infectious agents in the form of respiratory droplets and particles produced by the patient to flow away from the head of the patient and out of the transparent barrier while the medical personnel manages the airway and upper aerodigestive tract of the patient.

FIG. 4 illustrates a folded view of the metal frame used in the airway management isolation enclosure.

FIG. 5 illustrates an unfolded view of the metal frame used in the airway management isolation enclosure.

FIG. 6 shows an embodiment where the air flow entry and a vacuum exit connection are a separate unit that is supported on the frame of the airway management isolation enclosure. The air flow entry and vacuum exit are connected by flexible tubing.

FIG. 7A summarizes the air flow entry port, the vacuum exit port, the plurality of holes in the frame of the airway management isolation enclosure with air flow in and out of the frame.

FIG. 7B summarizes the air flow entry port, the vacuum exit port, the plurality of holes in the frame of the airway management isolation enclosure with air flow within the enclosure for removing infectious agents.

FIG. 8 summarizes the invented airway management isolation enclosure and the apparatus for testing ventilation of smoke particles.

FIG. 9 summarizes ventilation smoke test results for removal of particles from using a single barrier for the invented airway management isolation enclosure.

FIG. 10 summarizes ventilation smoke test results for removal of particles from using a double barrier for the invented airway management isolation enclosure.

FIG. 11 summarizes vacuum/air ventilation smoke test results for removal of particles from the invented airway management isolation enclosure.

FIG. 12 summarizes vacuum ventilation smoke test results for removal of particles from the invented airway management isolation enclosure.

FIG. 13 summarizes spacing vacuum ventilation smoke test results for removal of particles from the invented airway management isolation enclosure.

FIG. 14 shows an exemplary embodiment that the airway management isolation enclosure is useful for delivery of nebulized medications to the patient while protecting medical personnel from infectious agents.

### DETAILED DESCRIPTION

The present invention overcomes problems known in the art and associated with conventional enclosures for protecting patients from infecting medical personnel while performing medical procedures such as airway management. The advantages, and other features of the system disclosed herein, will become more readily apparent to those having ordinary skill in the art from the following detailed description of certain preferred embodiments taken in conjunction with the drawings which set forth representative embodiments of the present invention.

A major problem in hospitals, and particularly in emergency wards, is the risk of the medical personnel contracting contagious diseases from an infectious agents transmitted by a patient while performing medical procedures such as airway management on the patient. SARS-CoV-2 is an example of such an infectious agent that causes the disease COVID-19. COVID-19 is typically caused by spread of the virus in respiratory droplets and/or particles from an infected patient and the virus has been shown to persist in the environment for significant periods with up to 3 hours of viability in aerosols and up to 3 days on surfaces.

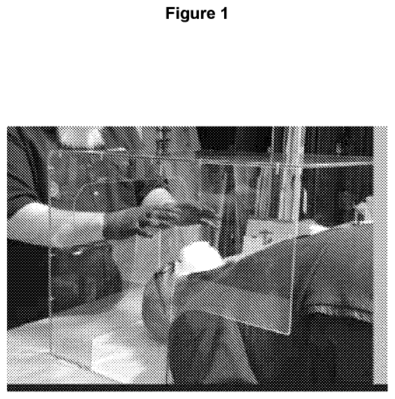

Conventional physical barriers disclosed in the relevant art (FIG. 1) provide minimal to no protection of medical personnel or healthcare workers from such infectious agents in the form of droplets or particles by infected patients that are generated at the time of definitive airway management (bagging, intubation). In addition, such physical barriers present problems to medical personnel including rigid holes that do not fit each healthcare professional and limit normal intubation mechanics, the fact that the physical barrier is open and does not seal off the infectious agents transmitted by the patient, and that limits space required for administering the airway management devices, (intubation equipment, laryngoscopes).

The invented airway management enclosure provides a solution for protecting medical personnel or healthcare workers while performing airway management procedures, such as high flow nasal cannula (when cannulization reaches a limit, such as 6 L/min and patient is still hypoxic, then patient is intubated in a controlled manner), nebulizer therapy, bilevel positive airway pressure (BiPAP), continuous positive airway pressure (CPAP) therapy. Prior to the inventors' discovery, COVID-19 therapy resulted in more intubations and significantly increasing mechanical ventilator usage. In fact, current COVID-19 therapeutic guidance significantly elevates airway management to minimize airborne and droplet production via early intubation.

The invented airway management isolation enclosure for protecting medical personnel from infectious agents when performing airway management or other medical procedures on a patient having an infectious disease, comprises: (a) a frame that is positioned over the head and anterior thoracic region of the patient; (b) a transparent barrier supported by the frame, which covers and forms a seal around the head and anterior thoracic region of the patient and which allows the medical personnel to create sealable entry ports; and (c) an air flow entry and a vacuum exit connection to direct the infectious agents in the form of respiratory droplets and particles produced by the patient to flow away from the head and out of the transparent barrier while the medical personnel manages the airway and upper aerodigestive tract of the patient.

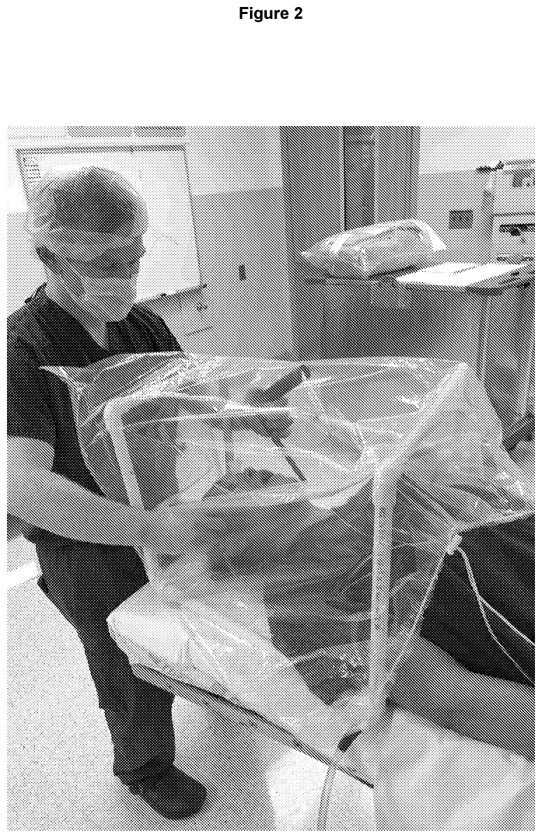

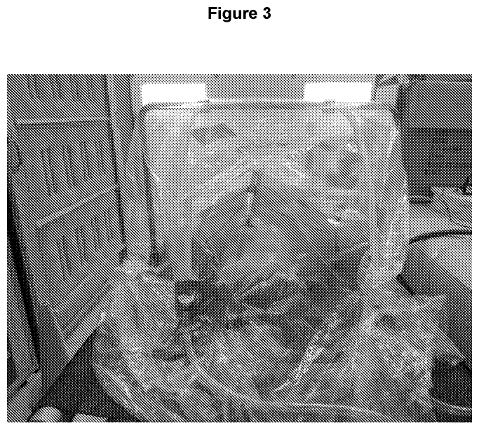

One embodiment of the invented airway management isolation enclosure using a plastic frame is summarized in FIG. 2. A separate embodiment of the invented airway management isolation enclosure using a metal frame is summarized in FIG. 3.

In one embodiment, the frame of the airway management isolation enclosure is hollow and comprises an air flow inlet connection port, a vacuum exit connection port which is connected to a vacuum source to create a negative pressure and a plurality of holes in the frame. The material of the frame can be fabricated from medical grade thermoplastics and polymers, as disclosed in U.S. Pat. No. 6,635,324 B1. In some embodiments, the frame can be manufactured from a reusable plastic, metal, ceramic, a composite or a combination of materials that can be decontaminated or sterilized. In some embodiments the isolation enclosure frame includes one or more air flow entry connections and one or more vacuum exit connections. In other embodiments, the frame is manufactured from a disposable plastic or composite material. In one embodiment the frame is a plastic material selected from medical grade polyvinylchloride (PVC), Teflon tubing, 3D printed carbon fiber and nylon12. In some embodiments, the frame can be manufactured from a reusable medical grade metal (e.g. stainless steel, aluminum, titanium) that can be decontaminated or sterilized. In some embodiments, other types of medical grade metals or ceramics can be used as frame materials. In some embodiments, the frame can be rigid or collapsible. In some embodiments, the frame can be modular or monolithic. In a separate embodiment, the airway management isolation enclosure comprises two or more frames, an outer frame and an inner frames one progressively and proportionally smaller than the outer frame or sequential smaller frames. The two or more frames accommodate two or more barriers, increasing the effectiveness of infectious agent removal while medical personnel perform air management on a patient.

In one embodiment, the airway management isolation enclosure is referred to as a COVID-19 Airway Management Isolation Chamber, or CAMIC. When used with other PPE, CAMIC protects health care personnel by providing a physical barrier to aerosolized droplets from patients with COVID-19 by capturing and removing viral particles emitted by the patient. The self-contained device centers around the head, neck, and torso of the patient, providing a barrier between patient and provider. The air/vacuum circulation ports are purposely positioned, tested and integrated directly into the CAMIC's support frame.

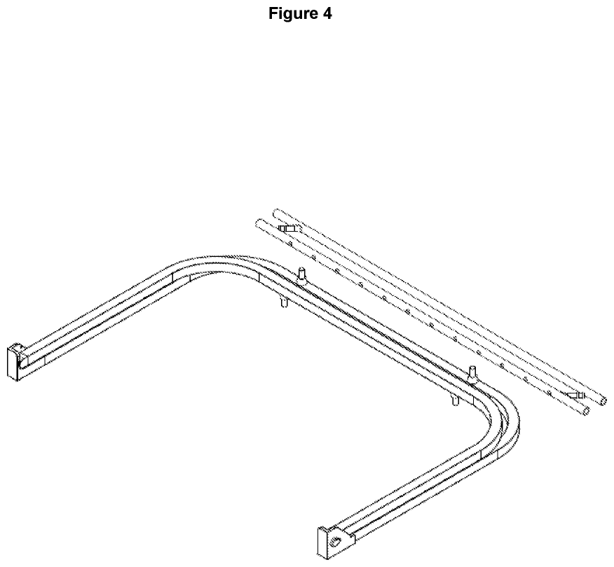

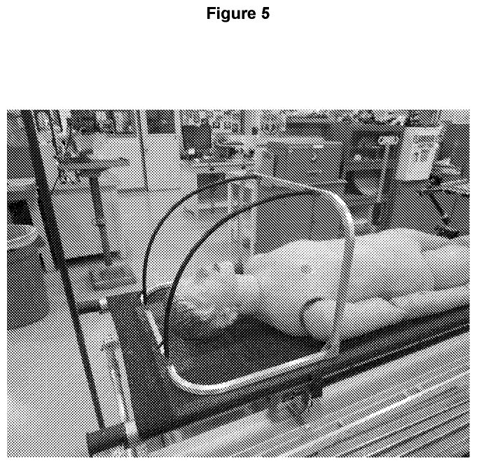

In one embodiment, the metal frame of the airway management isolation enclosure (CAMIC) is foldable (FIG. 4) and is unfolded when used (FIG. 5).

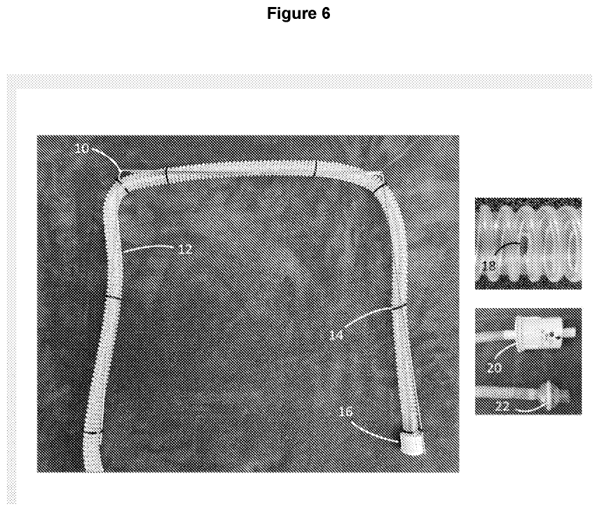

In a separate embodiment, the air flow entry and a vacuum exit connection are a separate unit that is supported on the frame of the airway management isolation enclosure. The frame supports tubing comprising a plurality of holes, an air flow entry connection and a vacuum exit connection, to direct air flow away from the head of the patient and out of the isolation enclosure. The air flow entry and vacuum exit are connected by flexible tubing (FIG. 6). FIG. 6 picture shows one half of the embodiment. Two parts are needed. One is for the inlet side that provides oxygen or air. The other is for the outlet side to capture/exhaust airborne particles along with expired CO₂ to a vacuum line equipped with HEPA filtration, The separate unit is usefully employed in accordance with other self-contained negative pressure environment devices (e.g, SCONE™). The metal frame member 10 is preferably made of stainless steel. Alternatively, this frame member could be omitted with tubing (12) attached directly to the frame of a device such as the SCONE™. The tubing 12 is supported on the metal frame 10 and is preferably CPAP tubing. The 19 mm inner diameter of CPAP tubing provides good flow with little pressure drop, it is widely available at low cost, and it is manufactured in accordance with FDA quality control standards. A tie 14 is used to secure tubing 12 to the metal frame 10, or alternative, to the frame of a device such as the SCONE™. A cap 16 is used to seal one end of tubing 12. The other end of tubing 12 (not shown) is attached to either a breathing gas source (pure oxygen or air) or a vacuum line. The vacuum line may be fixed as in, for example, those available in hospital rooms, or it may be provided by connection to a portable/cordless vacuum. An opening or aperture 18 is provided in a plurality of places along the length of tubing 12. The tubing is connected to a filter 20, which is one example of a HEPA filter that has a relatively long service life (e.g., months). Alternatively, a CPAP filter 22 is used with the appropriate filter media to provide N95 level protection. This low-cost type filter would be disposed after use and has a more limited service life compared to HEPA filter 20.

A transparent barrier of the airway management isolation enclosure is supported by the frame, which covers and forms a seal around the head and anterior thoracic region of the patient and which allows the medical personnel to create sealable entry ports. The barrier can be manufactured from a suitable medical grade thermoplastic or polymers, such as polyethylene, that is transparent and allows the medical personnel to create sealable entry ports. The barrier covering of the isolation can be disposable clear/translucent plastic (e.g., materials used for bags/trash bags or even such consumer products in an emergency) or a longer-lasting translucent vinyl or a similar thermoplastic material that is reusable and can be sterilized. In one embodiment, the medical personnel to create sealable entry ports in the barrier. In some embodiments, working ports (e.g., slits, magnetic slits, or sleeves) can be incorporated in the barrier on the chamber to facilitate procedures (such as intubation/extubation, surgery, and nursing/respiratory therapy care). In some embodiments, the barrier of the isolation enclosure frame includes one or more air flow entry connections and one or more vacuum exit connections.

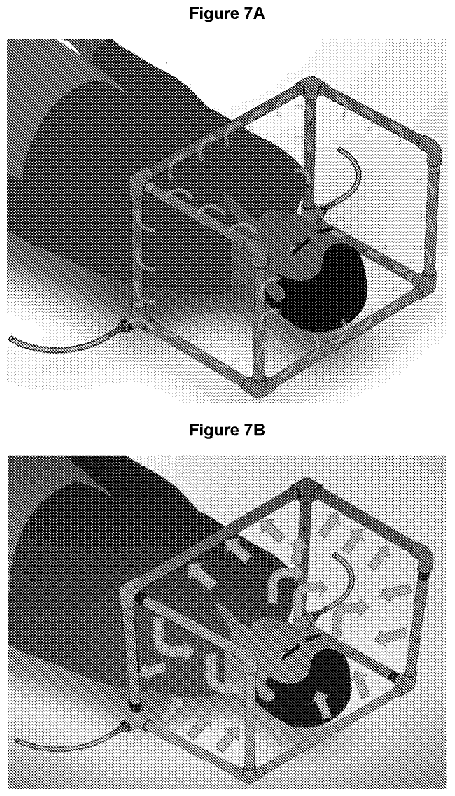

Infectious agents are removed from the airway management isolation enclosure by flowing air or oxygen (O₂) away from the head of the patient and out of the isolation chamber, while medical personnel perform airway management on a patient. In some embodiments, air flow direction with the isolation enclosure is effected by flowing air or O₂ though one or more entry connections and applying vacuum to one or more vacuum exit connections. In one embodiment, an entry port is provided within the frame connected to air or oxygen. A corresponding exit port is also provided in the frame which connects to a vacuum source. The relative negative pressure effect by vacuum moves infectious agents out of the isolation enclosure, as shown in FIG. 7, where the air flow in and out of the frame (1) and the air flow within the enclosure (2) is summarized. This air flow design eliminates or mitigates against eddies and/or dead spaces forming where virus particles could linger in the chamber as opposed to flowing out with the exhaust air. One advantage of the present invention is the air flow prevents eddies and/or dead spaces inside the chamber (e.g. in the corners) where air contaminated with virus particles could be trapped and even concentrated. Two ports are used in this embodiment of the invention. A first port is connected to a positive-pressure air or oxygen (air or O₂) source. A second port is connected to a vacuum source, which is further connected to a decontamination trap or exhaust system. This vacuum source may include one or more in-line HEPA filters through which the chamber exhaust flows before entering into a decontamination trap, room or building exhaust system for discharge to the atmosphere.

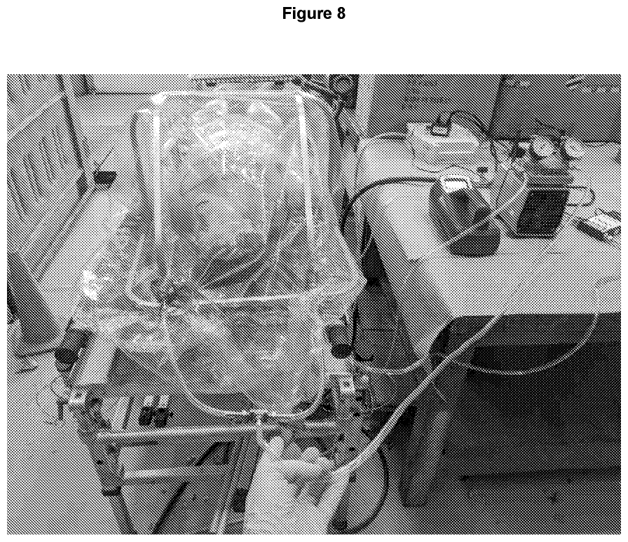

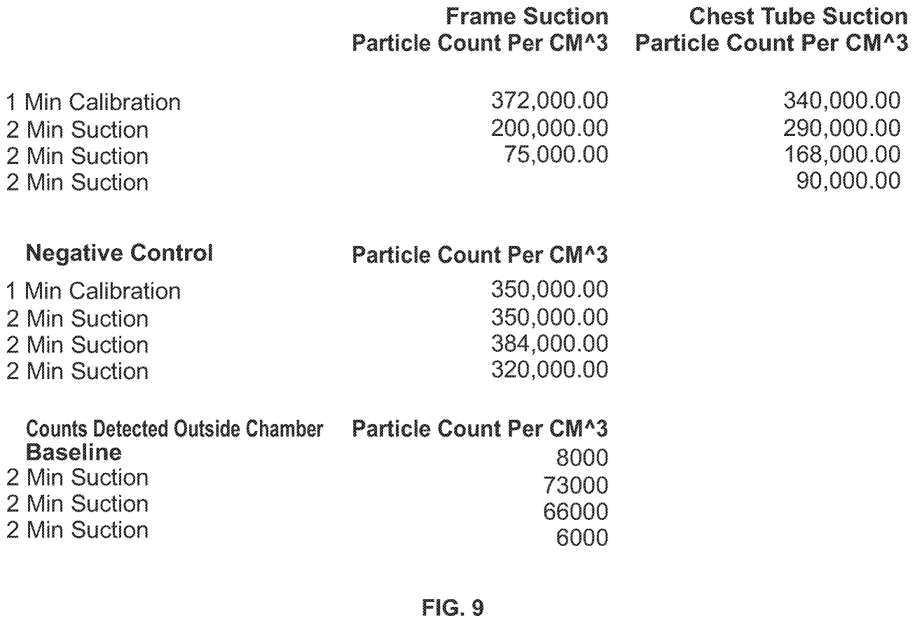

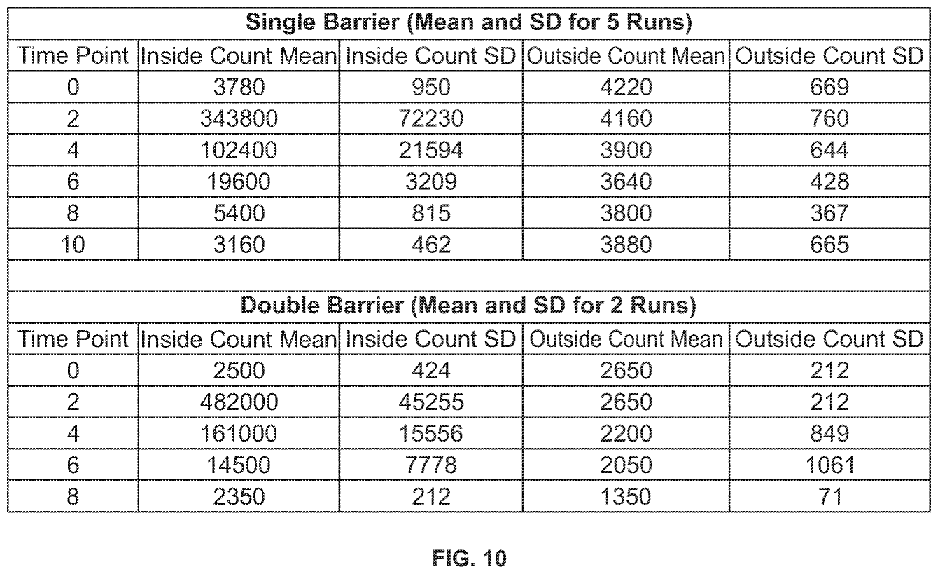

Ventilation smoke tests using a testing device outside the isolation chamber (FIG. 8) was conducted in a room of a medical device prototype fabrication facility adjacent to a room for painting which provided a slight flow of air in the room used for testing. A smoke model was used to obtain the data, and readings were taken as close to center of the chamber as possible, with the probe placed above the lips of the mannequin. It remained in a constant position during each variants test but position may vary between variants. Oxygen supply was simulated by using air from a compressor and regulating it to around 10 L/min which was measure by passing it through a calibrated flow rate sensor. Suction was simulated using a Gast air pump. It was set as close to 120 mmHg as could be estimated using the pump gauge. The gauge vibrated quite intensely during use but the adjustment knob was not changed during the course of testing so all CAMIC variants were tested under similar conditions with regard to suction supplied. Baseline room readings as well as a negative control were collected to determine the natural rate of dissipation of the particles. Ventilation smoke tubes were used containing ethylenediamine and acetic acid. Testing confirmed removal of particles from the invented airway management isolation enclosure are summarized in FIG. 9. Smoke particles were introduced in the enclosure and the smoke particles were measured using a particle counter (Condensation Particle Counter Model 3007) in the chamber and outside the chamber initially and after time intervals of 1 and 2 minutes. After two minutes of negative pressure within the isolation enclosure, a range of 73-80% decrease in particles was observed. Using a second barrier significantly improvement in the number of particles removed from the isolation enclosure, as compared to using a single barrier (FIG. 10). Ventilation smoke tests results demonstrates excellent decrease intra-chamber particulate counts and retention of particulate within chamber with negligible leak outside the device using both the single and double barrier method. There was less particulate matter outside of the bag using the double bag method.

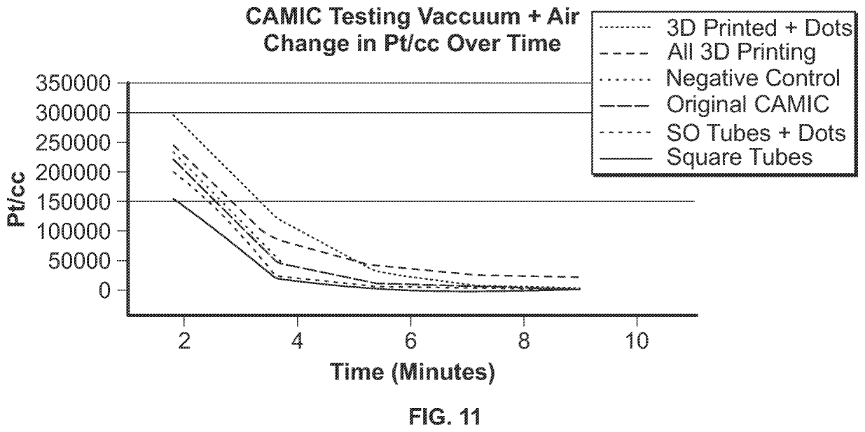

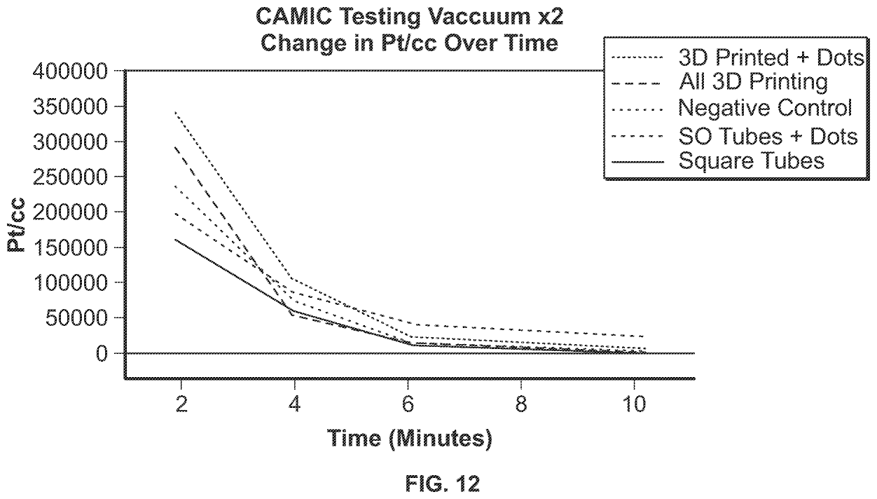

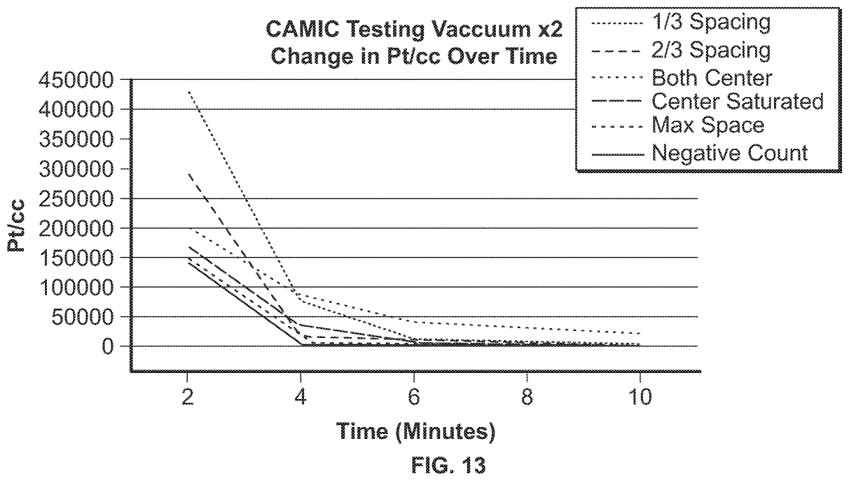

Vacuum and Air Testing
Two puffs were delivered from the ventilation smoke tube inside the CAMIC and two minutes of no suction/air were allowed to elapse before a reading was taken and the suction (˜120 mmHg) and Air (10 L/min) were initiated through the two ports leading to fenestrations on the CAMIC device. Suction on the variants had suction on the left and air supplied on the right ports. The original CAMIC had suction attached on the right and air supply the left. Given the symmetrical nature of the fenestrations in the CAMIC variants it is doubtful this had any dramatic effect but it has not been tested otherwise. Internal particle counts were taken every two minutes for 8 minutes. The results are summarized in FIG. 11. The graphing time axis starts at 2 minutes to account for the time when the suction/air were not in use. Internal Particle counts decreased at each time point.

Vacuum Only Testing
Two puffs were delivered from the ventilation smoke tube inside the CAMIC and two minutes of no suction was allowed to elapse before a reading was taken. Suction (˜120 mmHg) was then applied to both ports of the CAMIC. A splitter (seen in pic 5.) was used off of the main tube rather than a second supply. Internal particle readings we taken at two minutes after smoke was applied and in two minute intervals for 8 additional minutes. The graphing time axis starts at 2 minutes to account for the time when the suction/air were not in use. Internal Particle counts decreased at each time point.

CAMIC Spacing Testing (Vacuum and Air)
The CAMIC variant designated “3D Printed +COTS” was used for this iteration of testing. The curved tubing containing fenestrations are adjustable and were spaced at various distances to test if there was any difference in particle clearing based on this variable. For all but the last test two puffs were delivered from the ventilation smoke tube inside the CAMIC and two minutes of no suction/air were allowed to elapse before a reading was taken and the suction (˜120 mmHg) and Air (10 L/min) were started. Suction/air tubing placements were the same as in the “Vacuum and Air” tests. Internal particle counts were taken every two minutes for 8 minutes. One test was performed where the 10 puffs were administered to saturate the environment within the CAMIC with the tubing both at center. The graphing time axis starts at 2 minutes to account for the time when the suction/air were not in use. Internal particle counts decreased at each time point for all CAMIC variants and were all significantly lower than the negative control at 8 minutes of the CAMIC device being turned on. Regardless of starting value all Variants had a lower value within 4 minutes of the CAMIC device being turned on.

Infectious agents removed by the invented airway management isolation enclosure are pathogenic viruses and bacterial that can form aerosols or droplets, having a particle size from 0.002 to 2.00 microns.

In some embodiments, the infectious agents are viruses including:
Group I: viruses possess double-stranded DNA and include such virus families as Herpesviridae (examples like HSV1 (oral herpes), HSV2 (genital herpes), VZV (chickenpox), EBV (Epstein-Barr virus), CMV (Cytomegalovirus), Poxyiridae (smallpox) and many tailed bacteriophages. The mimivirus was also placed into this group.
Group II: viruses possess single-stranded DNA and include such virus families as Parvoviridae and the important bacteriophage M13.
Virion—Type of naked/Capsid nucleic Virus Family Virus Genus enveloped Symmetry add 1, Adenoviridae Adenovirus Naked Icosahedral, Papovaviridae Papillomavirus Naked Icosahedral, Parvoviridae B 19 virus Naked Icosahedral, Herpesviridae Herpes Simplex Virus, Varicella Enveloped Icosahedral ds zoster virus, Cytomegalovirus, Epstein Barr virus, Poxviridae Small pox virus, Vaccinia virus Complex Complex, Hepadnaviridae Hepatitis B virus Enveloped Icosahedral, Polyomaviridae Polyoma virus.

### RNA Viruses

Group III: viruses possess double-stranded RNA genomes, e.g. rotavirus.
Group IV: viruses possess positive-sense single-stranded RNA genomes. Many well known viruses are found in this group, including the picornaviruses (which is a family of viruses that includes well-known viruses like Hepatitis A virus, enteroviruses, rhinoviruses, poliovirus, and foot-and-mouth virus), SARS virus, hepatitis C virus, yellow fever virus, and rubella virus.
Group V: viruses possess negative-sense single-stranded RNA genomes. The deadly Ebola and Marburg viruses are well known members of this group, along with influenza virus, measles, mumps and rabies.
Virion—Type of naked/Capsid nucleic Virus Family Virus Genera enveloped Symmetry, Reoviridae Reovirus, Rotavirus Naked Icosahedral, Picornaviridae Enterovirus, Rhino virus, Naked Icosahedral Hepatovirus, Cardiovirus, Aphthovirus, Parechovirus, Erbovirus, Kobuvirus, Teschovirus, Caliciviridae Norwalk virus, Hepatitis E virus Naked Icosahedral, Togaviridae Rubella virus Enveloped Icosahedral, Arenaviridae Lymphocytic choriomeningitis Enveloped Complex, Retroviridae HIV-1, HIV-2, HTLV-I Enveloped Complex, Flaviviridae Dengue virus, Hepatitis C virus, Enveloped Complex ss Yellow fever virus, Orthomyxoviridae Influenzavirus A, Influenzavirus B, Enveloped Helical ss Influenzavirus C, Isavirus, Thogotovirus, Paramyxoviridae Measles virus, Mumps virus, Enveloped Helical ss Respiratory syncytial virus, Bunyaviridae California encephalitis virus, Enveloped Helical ss Hantavirus, Rhabdoviridae Rabies virus Enveloped Helical, Filoviridae Ebola virus, Marburg virus Enveloped Helical, Coronaviridae Corona virus, SARS-CoV-2, COVID-19, Enveloped Complex, Astroviridae Astrovirus Naked Icosahedral, Bornaviridae Borna disease virus Enveloped Helical.

### Reverse Transcribing Viruses

Group VI: viruses possess single-stranded RNA genomes and replicate using reverse transcriptase. The retroviruses are included in this group, of which HIV is a member.
Group VII: viruses possess double-stranded DNA genomes and replicate using reverse transcriptase. The hepatitis B virus can be found in this group.

In some embodiments, the infectious agents are bacterial pathogens that have been implicated in airborne transmission in health care facilities include, but are not limited to, group A streptococci, S. aureus, Neisseria meningitidis, and Bordetella pertussis. An outbreak of methicillin-resistant S. aureus in an intensive therapy unit was linked to the exhaust ducting of the adjacent isolation room ventilation system. Legionnaires' disease has occurred from exposure to aerosols generated from contaminated cooling towers. Additionally, the causative agent, Legionella pneumophila, has been isolated from aerosols produced by water faucets and shower heads, by humidifiers and nebulizers, and by squeezing manual ventilation bags.

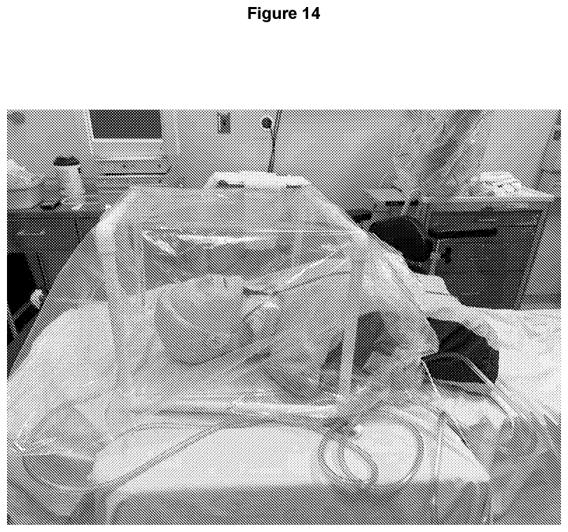

The invented airway management isolation enclosure has a number of advantages, namely reducing the need for immediate intubation and mechanical ventilator use. By decreasing the risk of exposure of medical personnel to infectious agents, the isolation enclosure allows for the use of aerosolizing procedures (such as nebulizers and positive airway pressure (BiPAP/CPAP) to be used for patients in respiratory distress, potentially removing or delaying their need for a ventilator. This is crucial during the COVID-19 pandemic. The device also allows clinicians to perform procedures through access ports while maximizing their protection as they work near the infected airway. The device can be manufactured with these access ports pre-established in the device or without. The latter option allows the healthcare worker using the device to cut access ports/slots in the desired location and length for their particular suitability and ease of performing a given medical procedure. FIG. 14 shows an exemplary embodiment that the airway management isolation enclosure is useful for delivery of nebulized medications to the patient while protecting medical personnel from infectious agents.

There are significant advantages of the invented airway management isolation enclosure: the elements of the isolation enclosure can be easily modified for situations where airway management are required (emergency room versus examination room) or by the requirements of the airway management equipment (e.g. laryngoscope, intubating equipment. The isolation enclosure allows airway specialist to make holes where needed to allow normal technique in management of airway. The isolation enclosure provides protection of medical personnel with the respect to intubation and extubation. The isolation enclosure provides airway management by mitigating the risk and enabling other procedures before intubation, such as nebulizer delivery of medicines, BiPAP and CPAP therapy, while protecting medical personnel associated with airway management of the patient. The mitigation avoids unnecessary or early intubations, reduces mechanical ventilator demand and may render ventilator use unnecessary for certain patients.

Other advantageous features on the invented isolation enclosure include: an optional disposable or easily decontaminated barrier; single or double barriers depending on the nature of the infectious agent; suction within the chamber to reduce the concentration of virus particles; optional second layer of suction between double barrier layers; flexibility to introduce air/oxygen into the chamber either through the frame itself or via a separate disposable component inserted into the chamber(s); the option to have working ports (e.g., slits, magnetic slits, sleeves) to facilitate treatments (by surgeons, doctors, nurses, respiratory therapists) or to allow the user to make such ports; various model designs specific for the working environment (i.e., larger width to facilitate intubation/extubation in the operating room/ICU) and smaller cubic chamber to allow for more air turnovers for better use in the Intensive Care Unit/Floor/Emergency Room where aerosol-generating procedures are more likely to be performed. Also, a smaller design could be used for patient transport or for use by Emergency Medical Services/Paramedics/Combat Medics to mitigate exposure in the field.

While specific aspects of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

1. An airway management isolation enclosure for protecting medical personnel from infectious agents when treating a patient having an infectious disease, comprising: (a) a frame that is positioned over the head and anterior thoracic region of the patient; (b) a transparent barrier supported by the frame, which covers and forms a seal around the head and anterior thoracic region of the patient and which allows the medical personnel to create sealable entry ports; and (c) an air flow entry and a vacuum exit connection to direct the infectious agents in the form of respiratory droplets and particles produced by the patient to flow away from the head of the patient and out of the transparent covering while the medical personnel manages the airway and upper aerodigestive tract of the patient.

2. The airway management isolation enclosure of claim 1, wherein the frame is hollow and further comprises a plurality holes and further comprises an air flow entry connection and a vac r exit connection, to direct air flow away from the head of the patient and out of the isolation enclosure.

3. The airway management isolation enclosure of claim 1, wherein the frame supports tubing comprising a plurality of holes, an air flow entry connection and a vacuum exit connection, to direct air flow away from the head of the patient and out of the isolation enclosure.

4. The airway management isolation enclosure of claim 1, wherein the vacuum exit further comprises a filter.

5. The airway management isolation enclosure of claim 1, wherein the frame is made from materials selected from stainless steel, aluminum, polyvinylchloride, carbon fiber and nylon-12.

6. The airway management isolation enclosure of claim 1, wherein the frame is modular.

7. The airway management isolation enclosure of claim 6, wherein the frame comprises polyvinylchloride (PVC) and is sterilizable.

8. The airway management isolation enclosure of claim 6, wherein the frame comprises stainless steel and is autoclavable.

9. The airway management isolation enclosure of claim 1, wherein the frame is portable.

10. The airway management isolation enclosure of claim 1, wherein the airway management isolation enclosure protects medical personnel while performing airway management from infectious agents selected from viruses possess positive-sense single-stranded RNA genomes, including the picornaviruses (which is a family of viruses that includes well-known viruses like Hepatitis A virus, enteroviruses, rhinoviruses, poliovirus, and foot-and-mouth virus), SARS virus, hepatitis C virus, yellow fever virus, and rubella virus, viruses possess negative-sense single-stranded RNA genomes, including Ebola and Marburg viruses, influenza virus, measles, mumps, rabies, Virion-Type of naked/Capsid nucleic Virus Family Virus Genera enveloped Symmetry acid, Reoviridae Reovirus, Rotavirus Naked Icosahedral, Picornaviridae Enterovirus, Rhino virus, Naked Icosahedral Hepatovirus, Cardiovirus, Aphthovirus, Parechovirus, Erbovirus, Kobuvirus, Teschovirus Caliciviridae, Norwalk virus, Hepatitis E virus Naked Icosahedral, Togaviridae Rubella virus Enveloped Icosahedral, Arenaviridae Lymphocytic choriomeningitis Enveloped Complex virus, Retroviridae HIV-1, HIV-2, HTLV-I Enveloped Complex, Flaviviridae Dengue virus, Hepatitis C virus, Enveloped Complex Yellow fever virus, Orthomyxoviridae Influenzavirus A, Influenzavirus B, Enveloped Helical ss Influenzavirus C, Isavirus, Thogotovirus, Paramyxoviridae Measles virus, Mumps virus, Enveloped Helical Respiratory syncytial virus, Bunyaviridae California encephalitis virus, Enveloped Helical Hantavirus, Rhabdoviridae Rabies virus Enveloped Helical, Filoviridae Ebola virus, Marburg virus Enveloped Helical, Coronaviridae Corona virus Enveloped Complex, SARS-CoV-2, COVID-19, Astroviridae Astrovirus Naked Icosahedral, Bornaviridae Borna disease virus Enveloped Helical, and bacterial pathogens.

11. The airway management isolation enclosure of claim 10, wherein the bacterial pathogens selected from group A streptococci, S. aureus, Neisseria meningitidis, Bordetella pertussis, and Legionella pneumophila.

12. A method for protecting medical personnel from infectious agents when performing airway management on a patient having an infectious disease by assembling an isolation enclosure that comprises the steps of: (a) positioning a frame over the head and anterior thoracic region of the patient laying in a supine position or a sitting position; (b) placing a transparent barrier supported by frame, which covers and forms a seal around the head and anterior thoracic region of the patient; (c) creating sealable entry ports in the transparent barrier; and (d) using a vacuum from an exit connection to direct the infectious agents in the form of respiratory droplets and particles produced by the patient to flow away from the head of the patient and out of the transparent barrier while the medical personnel manages the airway and upper aerodigestive tract of the patient.

13. The method of claim 12, positioning one or more progressive frames over the head and anterior thoracic region of the patient laying in a supine position or a sitting position in step (a); and placing a transparent barrier over each of the one or more frames in step (b).

14. The method of claim 12, wherein each frame or barrier comprises an entry air flow connect and a vacuum exit connection.

15. The method of claim 14, wherein the airway management isolation enclosure protects medical personnel while performing airway management from infectious agents selected from viruses possess positive-sense single-stranded RNA genomes, including the picornaviruses (which is a family of viruses that includes well-known viruses like Hepatitis A virus, enteroviruses, rhinoviruses, poliovirus, and foot-and-mouth virus), SARS virus, hepatitis C virus, yellow fever virus, and rubella virus, viruses possess negative-sense single-stranded RNA genomes, including Ebola and Marburg viruses, influenza virus, measles, mumps, rabies, Virion-Type of naked/Capsid nucleic Virus Family Virus Genera enveloped Symmetry acid, Reoviridae Reovirus, Rotavirus Naked Icosahedral, Picornaviridae Enterovirus, Rhino virus, Naked Icosahedral Hepatovirus Cardiovirus, Aphthovirus, Parechovirus, Erbovirus, Kobuvirus, Teschovirus Caliciviridae, Norwalk virus, Hepatitis E virus Naked Icosahedral, Togaviridae Rubella virus Enveloped Icosahedral Arenaviridae Lymphocytic choriomeningitis Enveloped Complex virus, Retroviridae HIV-1, HIV-2, HTLV-I Enveloped Complex, Flaviviridae Dengue virus, Hepatitis C virus, Enveloped Complex Yellow fever virus, Orthomyxoviridae Influenzavirus A, Influenzavirus B, Enveloped Helical ss Influenzavirus C, Isavirus, Thogotovirus, Paramyxoviridae Measles virus, Mumps virus, Enveloped Helical Respiratory syncytial virus, Bunyaviridae California encephalitis virus, Enveloped Helical Hantavirus, Rhabdoviridae Rabies virus Enveloped Helical, Filoviridae Ebola virus, Marburg virus Enveloped Helical, Coronaviridae Corona virus Enveloped Complex, COVID-19, Astroviridae Astrovirus Naked Icosahedral, Bornaviridae Borna disease virus Enveloped Helical, and bacterial pathogens.

16. The method of claim 15, wherein the bacterial pathogens selected from group A streptococci, S. aureus, Neisseria meningitidis, Bordetella pertussis, and Legionella pneumophila.
